(19) United States
(12) Patent Application Publication
(10) Pub. No.: US 2008/0200973 A1
(43) Pub. Date: Aug. 21, 2008

(54) Method and system using MRI compatibility defibrillation pads

(75) Inventors: Richard Philip Mallozzi, Ballston Lake, NY (US); Charles Lucian Dumoulin, Ballston Lake, NY (US)

Correspondence Address: GENERAL ELECTRIC COMPANY (PCPI);C/O FLETCHER YODER, P. O. BOX 692289, HOUSTON, TX, 77269-2289, US

(73) Assignee: General Electric Company

(21) Appl. No.: 11/708,420

(22) Filed: Feb. 20, 2007

(51) Int. Cl. A61N 1/05

(52) U.S. Cl. 607/142; 607/152

(57) ABSTRACT

In accordance with embodiments of the present technique an electrode pad for medical use is provides. The electrode pad comprises a support layer a plurality of electrodes mounted on the support layer and electrically insulated from one another, and a plurality of leads electrically coupled to the electrodes for selectively placing the electrodes at a desired electrical potential.

### BACKGROUND

The present invention relates generally to defibrillation systems. Particularly, the invention relates to defibrillation pads operable in conjunction with other medical systems during various medical procedures.

Defibrillation devices, otherwise known as defibrillators, are used to correct a medical condition known as fibrillation, which is a very rapid, disorganized twitching or trembling of the heart muscle in place of a normal rhythmic beat. To correct such a condition, a defibrillator directs a pulse of electrical direct-current (DC) into the heart to return it to its regular rhythm. To deliver such a pulse of electrical current to the heart of a patient, two defibrillation pads are attached, typically on the chest area of the patient. An electrical voltage applied between the defibrillation pads induces current through the heart of the patient, restoring the normal rhythm of the heart. Defibrillation pads are typically spread out in two dimensions, with typical lengths of several inches in each direction to provide a large contact area with the skin.

Various medical procedures may require coupling a patient to a defibrillator, via its defibrillation pads, as a precautionary measure. This may be done in order to expedite defibrillation therapy to the patient in the event the patient does experience fibrillation during the medical procedure. However, there are instances where the defibrillator pads can interfere with the medical procedure, such that it may not be operationally practical to couple the patient to the defibrillator. For example, during magnetic resonance imaging (MRI), a patient is placed within a partial enclosure whereby the patient is surrounded by static magnetic fields, dynamically-pulsed gradient magnetic fields, and radio frequency (RF) fields. These fields are used to interact with the atomic nuclei, exciting the population of magnetic moments and detecting microscopic magnetic fields induced by precessing nuclei. Electromagnetic interactions of the gradient and RF magnetic fields with various components of the defibrillation pads, e.g., wire leads and electrodes, may induce eddy currents that could interfere with imaging signals producing patient image data. To the extent such interference effects are present during the imaging procedure, they may create image artifacts and degrade image quality. Without a means to preserve image quality in the presence of defibrillation pads, it could become unfeasible to place such pads in the proximity of the MR imaging coils and expedite delivery of therapy in the event of urgent medical need.

There is a need in the art for improved defibrillation pads couplable to a patient during medical procedures. Particularly, there is a need for defibrillation pads couplable to a patient while the patient is situated within an MRI system such that the defibrillation pads minimally interfere with electromagnetic fields contained within the enclosure of the MRI system. There is also a need for similar pads that can be used during clinical interventional procedures such as cardiovascular ablation procedures.

### BRIEF DESCRIPTION

The present technique provides a defibrillation system based upon defibrillation pads couplable to a patient while the patient undergoes a medical procedure. In accordance with embodiments of the present technique, the defibrillation pads are operable within an imaging device such as an MRI device. Accordingly, the provided defibrillation pads and components thereof are configured to minimally interfere with electromagnetic signals produced by the MRI device. In this manner, image artifacts are minimized to the extent the images can provide desirable information relating to the patient to a clinician. Further, the present technique enables use of the defibrillation pads within the patient volume of the MRI device, thus eliminating time delays otherwise incurred in situations requiring exiting the patient from the MRI system before defibrillation pads can be applied to the patient.

The present technique further enables utilizing pads with similar geometry as defibrillation pads in other medical procedures, such as cardiovascular ablation procedures, whereby a conducting pad disposed on a patient provides an electrical ground connection for an ablation device.

### DRAWINGS

These and other features, aspects, and advantages of the present invention will become better understood when the following detailed description is read with reference to the accompanying drawings in which like characters represent like parts throughout the drawings, wherein:

FIG. 1 illustrates defibrillation pads disposed on a patient in accordance with an embodiment of the present technique;

FIG. 2 illustrates a segmented defibrillation pad in accordance with an embodiment of the present technique;

FIG. 3 is a side view of a packaged defibrillation pad in accordance with an embodiment of the present technique;

FIG. 4 is a side view of a defibrillation pad in accordance with an embodiment of the present technique; and

FIG. 5 illustrates defibrillation pads used in conjunction with a patient imaging device in accordance with an embodiment of the present technique.

### DETAILED DESCRIPTION

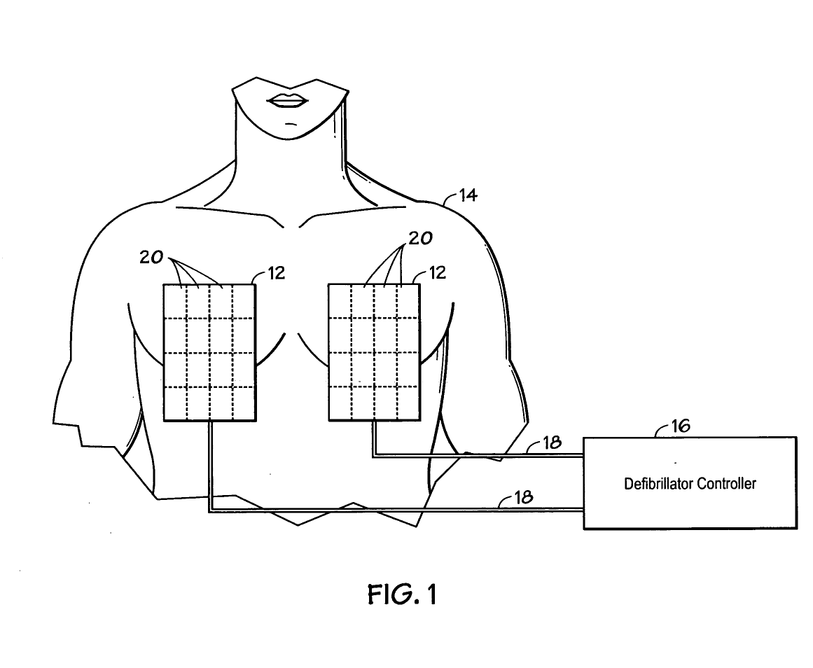

Turning now to the drawings and referring to FIG. 1, two defibrillation pads 12 disposed on a chest area of a patient 14 are shown, in accordance with an embodiment of the present technique. Each of defibrillation pads 12 are generally couplabe to a defibrillation control system 16 via wire lead 18. Defibrillation control system 16 may be a generic defibrillation system having connections compatible with multiple types and/or brands of defibrillation pads, such defibrillation pads 12. While the present embodiment illustrates two defibrillation pads coupled to control system 16, other embodiments may include more than two defibrillation pads, such as defibrillation pads 12, coupled to control system 16. Defibrillation pads 12 may be coupled to defibrillation control system 16 using plugs, clips, caps and so forth. Such coupling devices enable delivery of voltages and currents, such as those desired during defibrillation treatments, to and from pads 12. While in the illustrated embodiment, defibrillation pads 12 are disposed on the chest area of patient 14, in other embodiments the defibrillation pads may be configured to be disposed on, for example, a back side the patient or other suitable anatomical parts of patient 14. Accordingly, during, for example, a cardiac ablation procedure, one or more of the pads (although in such application not used for defibrillation) may be disposed on the patient so as to provide an electrical ground connection. In such a procedure, it may be desirable to place one of the defibrillation pads 12 on the back area of the patient.

As further discussed below, defibrillation pads 12 may be coupled to the patient via an adhesive and/or a gel that securely attaches defibrillation pads 12 to patient 14. Such an adhesive and/or gel may also be configured to conduct electric current between pads 12 and patient 14, thereby ensuring that a desirable level of current is delivered to the patient when voltage is applied to the defibrillation pads via defibrillation control system 16.

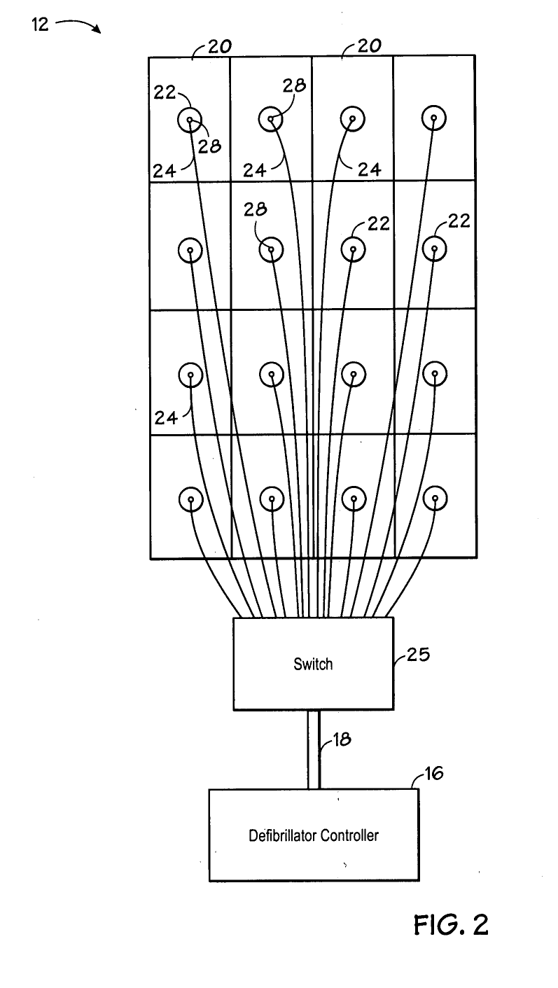

As depicted by FIG. 1, each of the defibrillation pads may be partitioned into segments 20, such that each segment may be separately couplabe to wire leads 18. Accordingly, each defibrillation pad 12 may be formed from individual segments 20 containing electrodes (FIG. 2), such that each electrode may be independently coupled to a voltage supply provided by defibrillation control 16. Hence, it should be borne in mind that wire leads 18, shown in FIG. 1, may actually be formed of strands of wire, such that each strand of wire leads to an electrode disposed within pad segments 20. As discussed below, the segmentation of the pads allows each pad to provide coverage and current comparable to conventional defibrillation pads, while limiting eddy currents within and around the pads due to the reduced size of each of the segments as compared to the overall size of the pad.

FIG. 2 illustrates schematically an implementation of a defibrillation pad, such as defibrillation pad 12 (FIG. 1), in accordance with an exemplary embodiment of the present technique. As depicted by FIG. 2, defibrillation pad 12 is segmented into multiple segments 20, such that each segment includes an electrode 22. Electrodes 22 may be formed of a conductive material, such as metallic foil, suitable for delivering electrical current to and from pad 12. Each of the electrodes 22 is separately coupled, via connection point 28, to a wire lead 24 independently coupling each of the electrodes to a voltage supply through a switch 25 which in some embodiments may be part of defibrillation controller 16. In the illustrated embodiment, switch 25 is separate from defibrillation controller 16 such that leads 24 are routed through the switch from which a single wire lead, such as one of wire leads 18, is provided to defibrillation controller 16.

Switch 25 is adapted to connect or disconnect each of leads 24 from defibrillation controller 16 so that, for example, when pads 12 are not in use, switch 25 (in an open state) electrically disconnects each of leads 24 from a power source, as well as from the other leads 24. Hence, switch 25 and the manner in which the defibrillation pads are segmented, as further described below, enables obtaining electrical configurations minimizing the extent to which eddy currents may interfere with surrounding electromagnetic fields about defibrillation pads 12 while the defibrillation system is idle. When the defibrillation system is in use, switch 25 is switched to a closed state, whereby each of leads 24 is placed at a desirable electrical potential and defibrillation pads 12 become electrically connected.

It should be borne in mind that the segmentation of pad 12 shown above is exemplary and that alternative segmentation patterns of pad 12 are possible, and within the scope of the invention, so as to accommodate various operational needs. For example, the number and shape of the segments may be varied to achieve the desired effects. That is, leads 18, 24 and electrodes 22 and the manner in which those elements are disposed throughout pads 12 and their segments 20 may determine the extent to which eddy currents produced by these elements influence a particular medical procedure in which the defibrillation pads are applied to the patient. Accordingly, certain medical procedures may require that defibrillation pads, such as defibrillation pads 12, be custom segmented in a manner which minimizes their interaction with the medical procedure, typically their interfering electromagnetic fields resulting from eddy current generation.

When the defibrillation pad 12 is in operation, each of the electrodes 24 is configured to sustain an electrical current such that the overall current delivered to or from the electrodes 22 conforms to a desirable electrical current used in defibrillation treatments. Further, partitioning pad 12 into individual segments, such as segments 20, reduces the overall magnitude of eddy currents produced by electrodes 22 and wire leads 24, and by the conductive components of the pads themselves. In other words, by segmenting the overall conductive area of each pad, connecting each electrode 22 separately to a voltage supply, and placing a plurality of such electrodes throughout pad 12, eddy currents due to changing magnetic fields are reduced. It should be borne in mind that the electrical configuration shown in FIG. 2, whereby each electrode 22 is separately coupled to a voltage supply via leads 24 is an exemplary configuration. As mentioned above, other electrical configurations of disposing and wiring electrodes 22 and leads 24 throughout pads 12 can be envisioned, resulting in an overall reduction of eddy currents across pads 12, as compared to conventional unsegmented pads. For example, it may be possible to connect all electrodes 24 disposed in a single row or a single column to a common lead, such that each row or column of pad 12 is separately connected to the voltage supply. This electrical configuration may, too, diminish eddy currents across the pad 12 which could optimally accommodate certain operational and/or clinical conditions arising in a specific medical procedure.

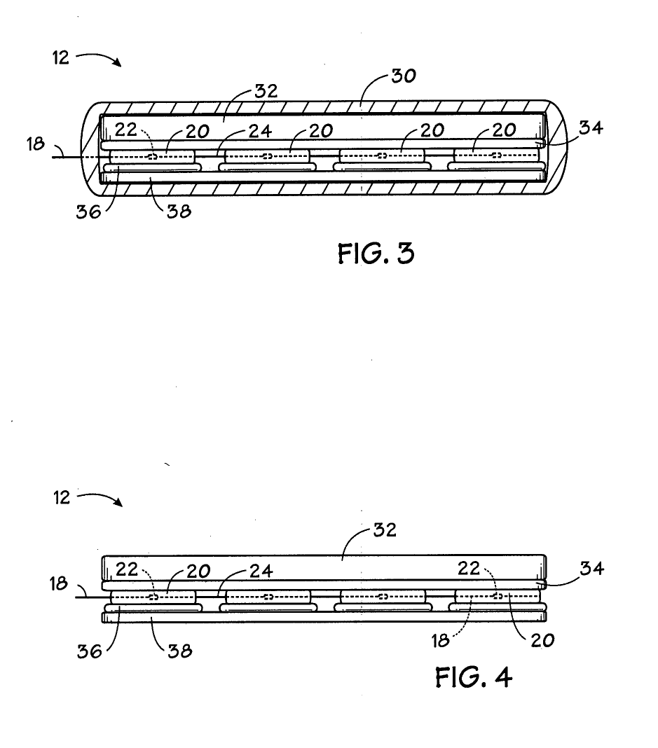

FIG. 3 is a side view of a packaged defibrillation pad, such as defibrillation pad 12, in accordance with an embodiment of the present technique. Accordingly, FIG. 3 depicts a packaged defibrillation pad, such as defibrillation 12, such that the pad is enveloped by a removable cover 30. Removable package 30 is configured to securely protect defibrillation pad 12 while the pad is stored and is not in use. Accordingly, package 30 may protect and preserve pad 12 from humidity or other corrosive elements or materials that otherwise would compromise electrical and/or mechanical components of the pad throughout its storage period. Package 30 may be formed of plastic, nylon, paper, styrofoam, combinations of these, or any other material that can be readily opened providing easy and fast access to pad 12. The package also may keep the pad sterile during transport and storage.

As further shown by FIG. 3, defibrillation pad segments 20, electrodes 22 and wire leads 24 are supported by a substrate 32 which may be formed of paper or a plastic material. Substrate 32 provides structural support for electrodes 22 and wire leads 24, as well as proper electrical insulation between electrodes 22 and leads 24. Substrate 32 may be coupled to pad segments 20, electrodes 22 and/or to wire leads 24 via an adhesive layer 34 disposed on the inner surface of substrate 32, i.e., the surface of substrate 32 facing electrodes 22 and wire leads 24.

Packaged defibrillation pad 12 further includes an adhesive layer 36 disposed on a side of the pad facing away from the electrodes 22 and wire leads 24. Accordingly, adhesive 36 is configured to securely affix pad 12, specifically electrodes 22, to the patient so as to ensure that pad 12 is retained on the patient for a prolonged period of time, as would be needed throughout a medical procedure in which defibrillation pads are employed. Further, adhesive layer 36 ensures that a suitable electrical contact exists between the patient and electrodes 22. Accordingly, adhesive layer 36 may be formed of materials having mechanical, electrical and/or thermal properties suited for interfacing between the defibrillation pads and the patient.

Defibrillation pad 12 further includes a gel layer 38 disposed over adhesive layer 36. Gel layer 38 is configured to enhance electrical coupling between the electrodes 22 and a patient to which pad 12 is applied. In other words, gel 38 may improve the electrical conductivity between the patient and the pad so as to better facilitate current flow to and from the pad during defibrillation. Such gels may be similar to those used conventionally on electrocardiograph and similar electrodes.

FIG. 4 is a side view of a defibrillation pad, such as the defibrillation pad 12 of FIG. 1, in accordance with an embodiment of the present technique. FIG. 4 illustrates an unpackaged defibrillation pad 12, ready for use as it would be applied to a patient. Accordingly, gel layer 38 provides an interface between pad 12 and the patient. As may be appreciated by those of ordinary skilled in the art, gel layer 38 may also be disposed on the patient before pad 12 is applied thereto. When applying pad 12 to the patient, pressing substrate 32 of pad 12 against the body of the patient may thin the gel layer 38 such that adhesive layer 36 may adhere pad segments 20 and, thus, electrodes 22 to the body of the patient.

As further illustrated by FIG. 4, wire leads 24 may extend throughout the pad 12 connecting each electrode, or alternatively a plurality of electrodes, to wire lead 18. Accordingly, wire leads 24 may be disposed along pad 12, providing sufficient slack to the extent pad segments 20 and substrate 32 can flex and conform to various curvatures and/or shapes of anatomical regions to which pad 12 is applied.

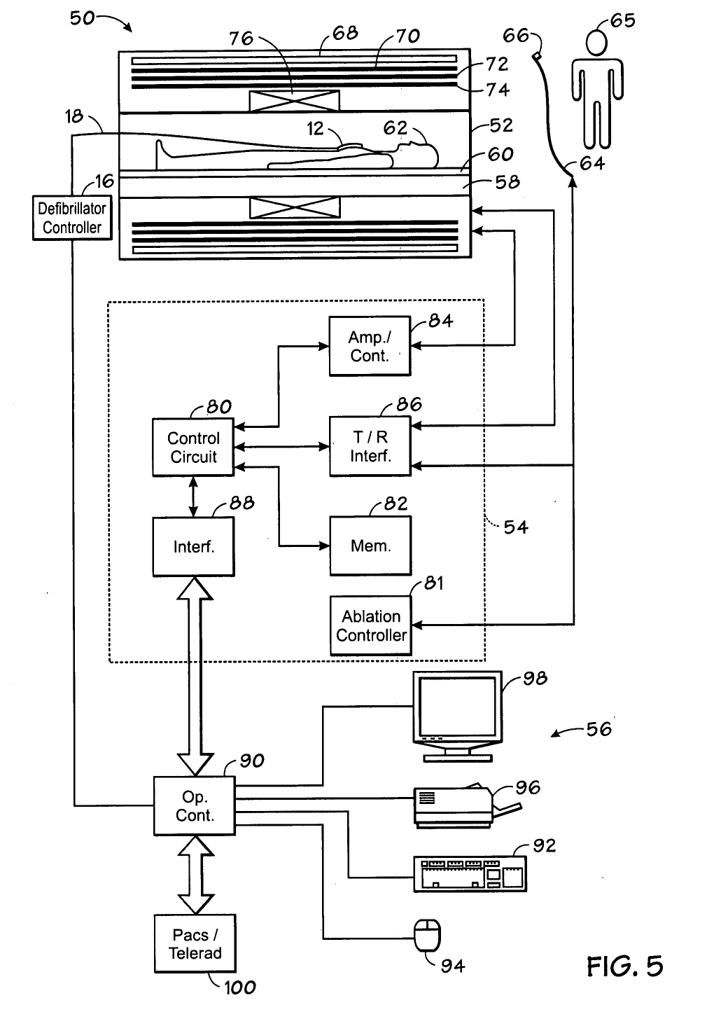

FIG. 5 is a diagrammatical representation of an imaging device such as an MRI system for use in medical diagnostic imaging and implementing use of a defibrillation system according to the present technique. The MRI system 50 suitable for MR diagnostic imaging and/or tracking is illustrated diagrammatically as including a scanner 52, scanner control circuitry 54, and operator interface station 56. While MRI system 50 may include any suitable MRI scanner or detector, in the illustrated embodiment the system includes a full body scanner comprising a patient bore 58 into which a table 60 may be positioned to place a patient 62 in a desired position for scanning.

As illustrated by FIG. 5, patient 62 is coupled to a defibrillation control system, such as the defibrillation system 16 shown in FIG. 1, via defibrillation pads 12 coupled to wire leads 18 connected the defibrillation control system. In the illustrated embodiment, the defibrillation pads are applied to the chest area of patient 62 undergoing imaging and/or tracking. Coupling patient 62 to a defibrillator while the patient undergoes a medical procedure, such as the one depicted by FIG. 5, may be desirable in an event the patient experiences fibrillation requiring defibrillation therapy. Under such conditions, defibrillation can be applied expeditiously while the patient remains in patient bore 58 such that no time is wasted on exiting the patient 62 from bore 58 before defibrillation can be performed.

As illustrated in FIG. 5, a device 64 to be tracked may be inserted into patient 62 by an operator 65. Device 64 may be any suitable device for use in a medical or surgical procedure. Device 64 may be a guide wire, a catheter, an endoscope, a laparoscope, a biopsy needle, an ablation device or other similar devices. For example, in an ablation procedure one pad, essentially identical to the defibrillation pads 12 discussed above, may be employed to electrically ground patient 62 so as to close an electrical loop with the ablation device applied to the patient. In such an embodiment, the grounding pad may be placed, for example, on the back area of the patient.

Further, non-invasive devices, such as external coils used in tracking, are also within the scope of the present embodiments. In such embodiments, device 64 may include an RF tracking coil 66 for receiving emissions from gyromagnetic material. Tracking coil 66 may be mounted, for example, in the operative end of device 64. Tracking coil 66 also may serve as a transmitting coil for generating radio frequency pulses for exciting the gyromagnetic material. Thus, tracking coil 66 may be coupled with driving and receiving circuitry in passive and active modes for receiving emissions from the gyromagnetic material and for applying RF excitation pulses, respectively. Hence, in a procedure utilizing RF tracking, wiring of defibrillation pads 12 may interact with the RF signals produced by the tracking the tracking device and RF coils so as to minimize generation of eddy currents.

Referring again to MRI system 50, scanner 52 includes a series of associated coils for producing controlled magnetic fields, for generating RF excitation pulses, and for detecting emissions from gyromagnetic material within the patient in response to such pulses. In the diagrammatical view of FIG. 5, a primary magnet coil 68 is provided for generating a primary magnetic field generally aligned with patient bore 58. A series of gradient coils 70, 72 and 74 are grouped in a coil assembly for generating controlled magnetic gradient fields during examination sequences. A radio frequency coil 76 is provided for generating RF pulses for exciting the gyromagnetic material. In the embodiment illustrated in FIG. 5, RF coil 76 also serves as a receiving coil. Thus, RF coil 76 may be coupled with driving and receiving circuitry in passive and active modes for receiving emissions from the gyromagnetic material and for applying radiofrequency excitation pulses, respectively. Alternatively, various configurations of receiving coils may be provided separate from RF coil 76. Such coils may include structures specifically adapted for target anatomies, such as head coil assemblies, and so forth. Moreover, receiving coils may be provided in any suitable physical configuration, including phased array coils, and so forth. The magnetic and RF fields produced by the gradient coils 70, 72 and 74 and the RF coil 76, respectively, interact with the defibrillation pads 12 to the extent eddy current produced by such interactions have no significant affect on gyromagnetic pulses obtained from the tissue of patient 62.

The coils of scanner 52 are controlled by external circuitry to generate desired fields and pulses, and to read signals from the gyromagnetic material in a controlled manner. As will be appreciated by those skilled in the art, when the material, typically bound in tissues of the patient, is subjected to the primary field, individual magnetic moments of the magnetic resonance-active nuclei in the tissue partially align with the field. While a net magnetic moment is produced in the direction of the polarizing field, the randomly oriented components of the moment in a perpendicular plane generally cancel one another. During an examination sequence, an RF frequency pulse is generated at or near the Larmor frequency of the material of interest, resulting in rotation of the net aligned moment to produce a net transverse magnetic moment. This transverse magnetic moment precesses around the main magnetic field direction, emitting RF (magnetic resonance) signals. For reconstruction of the desired images, these RF signals are detected by scanner 50 and processed. For location of device 64, these RF signals are detected by RF tracking coil 66 mounted in device 64 and processed. As mentioned above, the minimal interaction of the defibrillation pads 12 (FIG. 1), with the magnetic field and RF pulses produced by scanner 52 results in images having reduced artifacts that otherwise would be noticeable with conventional defibrillation pads used within an MRI system, such as that shown in FIG. 5.

Further, the coils of scanner 52 are controlled by scanner control circuitry 54 to generate the desired magnetic field and RF pulses. In the diagrammatical view of FIG. 5, control circuitry 54 thus includes a control circuit 80 for commanding the pulse sequences employed during the examinations, and for processing received signals. For example, control circuit 80 applies analytical routines to the signals collected in response to the RF excitation pulses to reconstruct the desired images and to determine device location. Control circuit 80 may include any suitable programmable logic device, such as a CPU or digital signal processor of a general purpose or application-specific determiner. Control circuitry further includes ablation controller 81 coupled to ablation device 64. Ablation controller 81 is configured to supply power to ablation device 64 so as to control voltage and current magnitudes used in ablation procedures. Control circuitry 54 further includes memory circuitry 82, such as volatile and non-volatile memory devices for storing physical and logical axis configuration parameters, examination pulse sequence descriptions, acquired image data, acquired tracking data, programming routines, and so forth, used during the examination sequences implemented by scanner 52. In the illustrated embodiment defibrillation controller 16 is shown as separate from control circuitry 56, however, in other embodiments the defibrillation controller may be included in control circuitry 54.

Interface between the control circuit 80 and the coils of scanner 52 and device 64 is managed by amplification and control circuitry 84 and by transmission and receive interface circuitry 86. Circuitry 84 includes amplifiers for each gradient field coil to supply drive current to the field coils in response to control signals from control circuit 80. Interface circuitry 86 includes additional amplification circuitry for driving RF coil 76. Moreover, where RF coil 76 serves both to emit the radiofrequency excitation pulses and to receive MR signals, circuitry 86 will typically include a switching device for toggling the RF coil 76 between active or transmitting mode, and passive or receiving mode. Interface circuitry 86 further includes pre-amplification circuitry to amplify the signals received by RF tracking coil 66 mounted in device 64. Furthermore, where RF tracking coil 66 serves as both a transmitting coil and a receiving coil, circuitry 86 will typically include a switching device for toggling RF tracking coil 66 between active or transmitting mode, and passive or receiving mode. Finally, circuitry 54 includes interface components 88 for exchanging configuration and image and tracking data with operator interface station 56. Hence, in situations, such as those described above, where RF signals are amplified or otherwise modified, wirings of wire leads 24 with electrodes 22 may be modified accordingly to obtain an electrical configuration of those elements which interact minimally with the RF and magnetic fields.

Operator interface station 56 may include a wide range of devices for facilitating interface between an operator or radiologist and scanner 52 via scanner control circuitry 54. In the illustrated embodiment, for example, an operator controller 90 is provided in the form of a determiner work station employing a general purpose or application-specific determiner. Operator controller 90 may be coupled to interface 88 of controller circuitry 54, as well as to defibrillator controller 16, so that an operator may monitor and control parameters pertinent to the mechanical procedure. The station also typically includes memory circuitry for storing examination pulse sequence descriptions, examination protocols, user and patient data, image data, both raw and processed, and so forth. The station may further include various interface and peripheral drivers for receiving and exchanging data with local and remote devices. In the illustrated embodiment, such devices include a conventional determiner keyboard 92 and an alternative input device such as a mouse 94. A printer 96 is provided for generating hard copy output of documents and images reconstructed from the acquired data. A determiner monitor 98 is provided for facilitating operator interface. In addition, system 50 may include various local and remote image access and examination control devices, represented generally by reference numeral 100 in FIG. 5. Such devices may include picture archiving and communication systems, teleradiology systems, and the like.

While only certain features of the invention have been illustrated and described herein, many modifications and changes will occur to those skilled in the art. It is, therefore, to be understood that the appended claims are intended to cover all such modifications and changes as fall within the true spirit of the invention.

1. An electrode pad for medical use, comprising:
a support layer;
a plurality of electrodes mounted on the support layer and electrically insulated from one another; and
a plurality of leads electrically coupled to the electrodes for selectively placing the electrodes at a desired electrical potential.

2. The pad of claim 1, comprising an adhesive region on or adjacent to the electrodes for adhesively securing the pad to the skin of a patient.

3. The pad of claim 1, comprising a conductive gel disposed over the electrodes.

4. The pad of claim 1, wherein the electrodes are made of a metallic foil.

5. An electrode pad kit for medical use, comprising:
a support layer;
a plurality of electrodes mounted on the support layer and electrically insulated from one another;
a plurality of leads electrically coupled to the electrodes;
means for coupling the leads to an electrical system to selectively place the electrodes at a desired electrical potential;
an adhesive region on or adjacent to the electrodes for adhesively securing the pad to the skin of a patient; and
a removable layer disposed over the electrodes and the adhesive region.

6. The kit of claim 5, comprising a conductive gel disposed over the electrodes.

7. The kit of claim 5, wherein the electrodes are made of a metallic foil.

8. A defibrillator system comprising:
a plurality of defibrillator pads, each pad including a support layer, a plurality of electrodes mounted on the support layer and electrically insulated from one another, and a plurality of leads electrically coupled to the electrodes, the pads being configured to be removably secured to the skin of a patient; and
a defibrillator controller coupled to the electrodes and configured to selectively place the electrodes of each pad at a desired potential for causing a current to flow through the patient.

9. The system of claim 8, wherein each pad includes an adhesive region on or adjacent to the electrodes for adhesively securing the pad to the skin of a patient.

10. The system of claim 8, wherein each pad includes a conductive gel disposed over the electrodes.

11. The system of claim 8, wherein the electrodes are made of a metallic foil.

12. The system of claim 8, comprising a switch coupled to at least one of the defibrillation pads and to the defibrillation controller, wherein the switch is configured to electrically connect and disconnect each one of the plurality of leads to and from a power source.

13. A method for imaging a patient anatomy comprising:
securing an electrode pad to a patient, the pad comprising a support layer, a plurality of electrodes mounted on the support layer and spaced from one another, and a plurality of leads electrically coupled to the electrodes for selectively placing the electrodes at a desired electrical potential;
placing the patient in an MR imaging system;
controlling the MR imaging system to produce image data for reconstruction of anatomies of interest within the patient.

14. The method of claim 13, comprising securing a plurality of similar electrode pads to the patient, and electrically coupling the electrode pads to a defibrillation controller.

15. The method of claim 14, wherein the pad includes an adhesive region on or adjacent to the electrodes for adhesively securing the pad to the skin of a patient.

16. The method of claim 14, wherein the pad includes a conductive gel disposed over the electrodes.

17. The method of claim 14, wherein the electrodes are made of a metallic foil.

18. A cardiac ablation system comprising:
a catheter insertable into a patient and having a conductive tip for applying a first desired potential to tissues of interest for ablation;
a conductive pad including a support layer, a plurality of electrodes mounted on the support layer and spaced from one another, and a plurality of leads electrically coupled to the electrodes for applying a second desired potential to the electrodes, the pad being configured to be removably secured to the skin of a patient; and
an ablation controller coupled to the catheter and to the electrodes and configured to selectively place the catheter tip and the electrodes of the pad at the first and second desired potentials for ablation of the tissues of interest.

19. The system of claim 18, comprising a device to be tracked coupled to the catheter.

20. The system of claim 19, wherein the device to be tracked is tracked via an imaging device.

21. The system of claim 20, wherein the imaging device is a magnetic resonance scanner.
